(19) United States
(12) Patent Application Publication
(10) Pub. No.: US 2009/0246813 A1
(43) Pub. Date: Oct. 1, 2009

(54) SELECTIVE ACCELERATION OF FRAGMENTATION THROUGH JOINT APPLICATION OF ENZYMES AND ULTRASOUND

(75) Inventors: Jose Luis Capelo Martinez, Monte da Caparica (PT); Jesus Vazquez Cobos, Madrid (ES); Daniel Lopez Ferrer, Madrid (ES); Isabel Moura, Monte da Caparica (PT)

Correspondence Address: SUGHRUE MION, PLLC, 2100 PENNSYLVANIA AVENUE, N.W., SUITE 800, WASHINGTON, DC, 20037, US

(73) Assignees: FACULDADE DE CIENCIA E TECHNOLOGIA-UNIVERSIDADE NOVA DE LISBOA, MONTE DE CAPARICA (PT); CONSEJO SUPERIOR DE INVESTIGACIONES CIENTIFICAS, MADRID (ES)

(21) Appl. No.: 11/997,876

(22) PCT Filed: Jul. 7, 2006
(86) PCT No.: PCT/IB06/52314

§ 371 Date: May 27, 2009

(30) Foreign Application Priority Data
Jul. 7, 2005 (PT) 103303

(51) Int. Cl. C12Q 1/40; C12Q 1/37

(52) U.S. Cl. 435/22; 435/23

(57) ABSTRACT

The present invention refers to an acceleration process for macromolecular fragmentation in liquid medium, in gel or in a solid support, by means of joint application of enzymes and ultrasounds, as well as isotopes. The macromolecules, in any of these liquids, are mixed with enzymes and inserted in a recipient (2) and exposed to an ultrasonic radiation field (1). The combined effect of the enzymes and of the ultrasounds allows accomplishing, in time ranges between 10 s and 600 s, the fragmentation of macromolecules or whole proteontes, with the correspondent formation of their constituent lower molecular units.

### OBJECT

The present invention describes a way for accelerating chemical, pharmaceutical, medical, biotechnological, or industrial procedures entailing protein or peptide fragmentation through enzymes for subsequent protein or peptide identification through protein or peptide identification-based techniques. The object is to accelerate the interaction of enzymes with proteins or peptides of natural origin, or polymers, or natural or biological macromolecules prepared by genetic engineering, or proteins, from the usual period of hours, to seconds or minutes. The object is attained throughout the use of ultrasound of frequency in the range of 10 to 100 kHz in contact with the enzymes and the substrate containing the proteins or peptides.

### STATE OF THE ART

The use of enzymes with the object of studying biological mechanisms has a broad tradition in science [Steen, H., Mann, M., Nat. Rev. Mol. Cell. Bio. 2004, 5, 699-71]. The study of the physical and chemical organization, as well as the identification and quantification of the proteomes' dynamic in living organisms ought to be as effective as possible. Therefore, it is important to develop new methodologies allowing the fast identification and quantification of a great number of proteins. Protein identification is carried out through the previous separation of the proteins and through the subsequent protein degradation in their constituent peptides, with the consequent peptide identification, through which the protein identification is achievable.

The complex protein mixtures are separated, by means of:

(1) One or two dimensional polyacrilamide gel electrophoresis. With this procedure the proteins are separated into bands. Subsequently, the bands containing the proteins are cut, separated and removed from the gel and exposed to enzymatic digestion. The peptides resulting from the protein decomposition, thus obtained, are analyzed by different techniques, including peptide separation and identification.

(2) Other alternative is the direct digestion of a pool of proteins in a liquid medium through the use of enzymes, with the subsequent formation of their constituent peptides. Afterwards, the peptides are separated and analysed, in-line through two-dimensional liquid chromatography.

The procedures for the protein separation or for their degradation into the constituent peptides are complex and time-consuming. The most time-consuming step of the process is the enzymatic digestion of the protein. The complete protocol for the enzymatic digestion of proteins can take as much time as 12 h [Steen, H., Mann, M., Nat. Rev. Mol. Cell. Bio. 2004, 5, 699-711].

The enzymatic digestion can be achieved by processes well known to the persons skilled in the art, including, for example, the ones described in Havlis, J., Thomas, H., Sebela, M. and Shevchenko, A., Anal. Chem. 2003, 75, 1300-1306, or in López-Ferrer, D., Martinez-Bartolome, S., Villar, M., Campillos, M., Martin-Maroto, F., Vázquez, J., Anal. Chem., 2004, 76, 6853-6860.

The works cited in the foregoing paragraph present a notion of the methodologies used for the degradation of macromolecules. The most common techniques use heating at 37° C. during 12 h, or heating at 60° C. during 30 min, the usage of organic solvents or detergents, or the acceleration of the process by the use of a microwave oven. The new proposed process has the following main advantages: (i) it is the fastest method known until now, (ii) it is the system with the highest throughput per time unit and (iii) it is a technology of easy access, handling and implementation.

In science, ultrasounds have been used for a long time for the degradation of elements. Due to this fact, no one has tried before to combine the enzymes with the ultrasounds, given the possibility of protein or peptide fragmentation. Other uses of ultrasound and enzymes are described in document PCT/IL2004/000104 which discloses a method for enhanced chemical debridement. However, this document does not disclose the rapid protein/peptide degradation for subsequent protein/peptide identification by protein/peptide identification-based techniques as it is in the present invention.

Document “Ultrasound enhancement of fibrinolysis at frequency at frequencies of 27 to 100 kHz” of Suchkova V. et. al (Ultrasound in Medicine and Biology, vol. 28, no 3, March 2002) discloses the use of ultrasound to accelerate enzymatic fragmentation of macromolecules in a liquid medium, particularly to enhance fibrinolysis, and its application to thrombolisys therapy. This document does not disclose or focus the use of a combination of enzymes and ultrasound to fasten the degradation of protein and/or peptide for subsequent identification and quantification by common identification-techniques as it is proposed in the present invention.

Document WO2004/069147 discloses a system and a method for enhancing the functionality and efficiency of a chemical and/or enzymatic debriding agent, with application of a combination of that debriding agent with ultrasound. However, it does not teach or suggest the process for protein or peptide identification and quantification by applying a combination of enzymes and ultrasound followed by the use of common identification-base techniques as it is disclosed in the present invention

Therefore, the closest related background art does not teach or suggest the combination of enzymes and ultrasound for accelerating the processes for protein or peptide fragmentation with the aim of protein or peptide identification through protein or peptide identification-based techniques. The background art also does not teach or suggest the combination of the ultrasound or the enzyme for accelerating the protein or peptide fragmentation for subsequent application for protein or peptide identification.

The present invention overcomes these deficiencies of the background art by describing a method for enhancing the functionality and efficiency of the methodologies using enzymes for protein or peptide fragmentation with subsequent protein or peptide identification, by means of preferably but not limited to mass spectroscopy techniques.

### DESCRIPTION OF THE INVENTION

The procedure consists in mixing the macromolecules, either in solution or in solid support, with the enzymes. Afterwards, the ultrasounds are applied to accelerate the enzymatic degradation. The ultrasounds can be applied by methods such as sonoreactors, ultrasound probes or ultrasound baths.

The physical and chemical properties of enzymatic and chemical reactions are greatly modified with the presence of an ultrasonic field. The mechanisms by which the enzymatic reactions are accelerated by means of ultrasound are due to:
(1) the diffusion coefficients are accelerated because ultrasounds 1) increase the temperature of the medium in which they are applied, which increases the reaction velocity, 2) by agitation of the material present in the solution, the enzymes become a stronger contact with the present substances, causing a larger total contact surface. It must be remarked that ultrasounds reduce the size of the solids presents in solution, incrementing the available contact areas.
(2) the penetration capability of ultrasonics, along with their capability of perforating soft material, such as biological tissues, turns ultrasound into an ideal tool for breaking and separating proteins from solids, such as gels or cellular membranes. Once the proteins have been separated, and already in contact with the enzymes, the digestion is accelerated by the ultrasounds as described in (1).

The enzymatic digestion of proteins and peptides accelerated with ultrasound is useful in all the scientific research areas in which it is necessary to apply the enzymes over biological substrates of natural origin, or over polymers or natural or biological macromolecules produced by genetic engineering, with the aim of: i) protein or peptide identification by mass spectrometry techniques, by identification, ii) being applied for pharmaceutical, biochemical, medical, chemical, mathematical or biological studies or for the treatment of diseases, such as Parkinson, affective disorders of the brain and modification of the nerve function in degenerative diseases, disorders of the peripheral catecholaminergic transmission, namely arterial hypertension, intestinal malabsorption syndrome, pathology of gastric and duodenal ulcer, renal function disorders, or in the study of any type of cancer.

Processes for preparation of samples with origin in any living organism for medical, pharmaceutical, mathematical, biotechnological or biological research purposes, characterized by: resulting the reactions from the application of the enzymatic reactions accelerated by ultrasounds.

Enzymatic reactions characterized by; i) being applied over polymers, natural or biological macromolecules produced by genetic engineering.

The described processes allow macromolecular fragmentation of any complex mixture of proteins, whole proteome, protein, or peptides in their lower components that are then used for protein or peptide identification by preferably but not limited to, mass spectrometry techniques. Table 1 presents results for the accelerated fragmentation of different proteins as result of the combination of ultrasound and the enzyme trypsin, and the subsequent identification by mass spectrometry techniques.

### DETAILED DESCRIPTION OF THE INVENTION

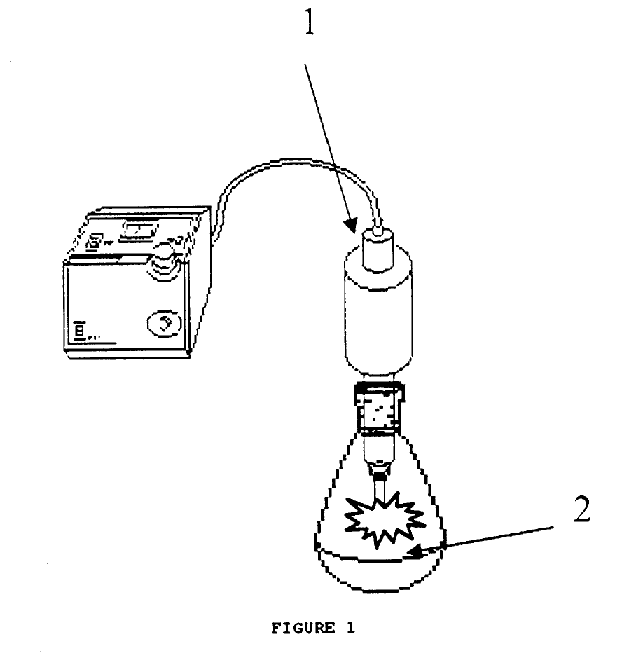

The present invention refers to the supply system of a high or low frequency ultrasound system. In FIG. 1 is presented an ultrasound probe (1). Other systems for supplying ultrasound can also be used, such as external ultrasound transducers, resonating tube reactors and submersible transducers and, in a general way, any method for supplying ultrasound, such as the ones described in: T. J. Mason, Sonochemistry, Oxford University Press, New York, 1999.

The container can contain any kind of enzyme (2), and any biological substrate of natural origin, or any polymer or natural or biological macromolecule prepared by genetic engineering, or any isotope.

When the sonication process with the material referred to in point 2 takes place, the referred material will be decomposed in its minor constituents as a consequence of the enzymatic action which is accelerated by the action of the ultrasounds. This acceleration allows a time reduction from hours to seconds or minutes. Thus, a protein will decompose in peptides or even smaller organic molecules. The atoms interchange by their isotopes is thus also accelerated, being the ¹⁶O substituted by ¹⁸O. Then the fragments obtained are used for protein or peptide identification through, for e.g. by mass spectrometry techniques.

Table 1 presents data obtained from a protein fragmentation through the combination of ultrasonic probes and the enzyme trypsin with subsequent protein identification by matrix assisted laser desorption ionization mass spectrometry technique. Sonication period 120 s sonication amplitude 70%, probe diameter 0.5 mm. Trypsin concentration 14.4 μg/ml. Fragmented protein mass: 1.7 μg of phosphorylase b, 2.1 μg of albumin, 3.7 μg of ovalbumin, 2.1 μg of carbonic anhydrase. The proteins were separated by gel electrophoresis and the fragmentation of the proteins was realized in-gel. The fragmentation was used for the protein identification by MALDI-TOF-MS.

When enzymes and elemental isotopes, individually or in molecules, are mixed together, the described procedures allow the degradation of any organic components from living organism in short periods, from 10 to 600 s, in their correspondent proteins, and, then, the proteins in their correspondent components, and interchanging elements by their correspondent isotopes.

TABLE 1

| | | | | MASCOT | | |
|---|---|---|---|---|---|---|
| | V | Peptides | Inten- | | Cover | Identified |
| Protein | (μl) | (fragments | sity | Scores | (%) | peptides |
| Phosphor- | 25 | 37 | 1.8E5 | 78 | 12 | 13 |
| ylase b | | | | | | |
| Phosphor- | 100 | 43 | 2.4E5 | 256 | 33 | 27 |
| ylase b | | | | | | |
| Albumin | 25 | 29 | 2.8E5 | 108 | 21 | 12 |
| Albumin | 100 | 26 | 9.8E4 | 172 | 24 | 16 |
| Ovalbumin | 25 | 28 | 8.3E4 | 90 | 30 | 8 |
| Ovalbumin | 100 | 36 | 9.4E4 | 108 | 36 | 10 |
| Carbonic | 25 | 35 | 2.9E5 | 203 | 59 | 14 |
| anhydrase | | | | | | |
| Carbonic | 100 | 28 | 2.6E5 | 142 | 54 | 10 |
| anhydrase | | | | | | |

1.-8. (canceled)

9. Process for protein and/or peptide identification and quantification characterized for co-applying ultrasounds and enzymes followed by the use of an identification and quantification-protein/peptide technique, comprising:
a) adding an enzyme or a mixture of enzymes to a given biological sample;
b) exposing the biological mixture to an ultrasound field;
c) analyzing the mixture with fragmented proteins and/or peptides with the identification and quantification-protein/peptide technique.

10. Process for protein and/or peptide identification and quantification, according to claim 9, wherein the enzymes present in a mixture comprise the following families: lipases, amylases, or proteases.

11. Process for protein and/or peptide identification and quantification, according to claim 9, wherein the applied ultrasounds are supplied by an ultrasound probe, an ultrasound bath or a sonoreactor or any other means for producing an ultrasound field.

12. Process for protein and/or peptide identification and quantification, according to claim 9, wherein the identification and quantification-protein technique comprises mass spectrometry.

13. Process for protein and/or peptide identification and quantification, according to claim 9, comprising adding isotopes to the biological mixture.

14. Use of the process for protein and/or peptide identification and quantification, according to claim 9, being applied over proteins and peptides present in biological samples in liquid medium, solid support, in a gel or in solid form, to originate the selective fragmentation of the protein or the proteins in peptides, organic or inorganic molecules for the subsequent protein or peptide identification and quantification.

15. Use of the process, according to claim 14, wherein the subsequent protein or peptide identification and quantification comprises mass spectrometry.
